(19) United States
(12) Patent Application Publication
(10) Pub. No.: US 2010/0228200 A1
(43) Pub. Date: Sep. 9, 2010

(54) DOSAGE DELIVERY DEVICE

(76) Inventor: Paul Harry Moed, St. Peter Port Guernsey (GB)

Correspondence Address: DAVID A. GUERRA;INTERNATIONAL PATENT GROUP, LLC, Suite 700, 1816 Crowchild Trail N.W., CALGARY, AB, T2M 3Y7, CA

(21) Appl. No.: 11/993,402

(22) PCT Filed: May 12, 2006
(86) PCT No.: PCT/GB06/01743

§ 371 Date: Dec. 20, 2007

(30) Foreign Application Priority Data
Jun. 24, 2005 (GB) 0512893.9
Mar. 6, 2006 (GB) 0604420.0

(51) Int. Cl. A61M 5/315; A61M 5/31

(52) U.S. Cl. 604/192; 604/218

(57) ABSTRACT

A dosage delivery device, for example a syringe type device, having a housing (2) defining a reservoir (4); a plunger (6) within the reservoir and movable to decrease the volume of the reservoir so as to discharge material in the reservoir via a discharge opening; and an operating mechanism for the plunger, wherein a closure member (12) seals the discharge opening, and is coupled to the plunger operating mechanism such that it exposes the opening when the mechanism is engaged. In this way, a single movement to prime the mechanism also removes the closure.

This invention relates to dosage delivery devices and particularly but not exclusively, to such devices for use in the oral and anal delivery of medicaments. Such devices are of particular value in emergency situations where a dosage of medicament has to be delivered urgently and with minimal preparation of the device prior to use.

Various modern medicines are adapted for oral or anal delivery, and in prescribed quantities. They can be provided in capsule form, sometimes with the capsule material being soluble so that it dissolves in the user's mouth or anus. Dosages can also be delivered by aerosol and for dosages in liquid form particularly can be delivered by means of a pump or syringe. Devices of this latter type would include a reservoir with a discharge opening at one end, with a plunger in the reservoir being movable towards the opening to discharge liquid in the reservoir through the opening. The present invention is directed at devices of this type.

In order to move the plunger in the reservoir of a delivery device of the kind just referred to, an actuating member has to extend beyond the reservoir by at least the full stroke of the plunger when delivery commences. If the device is pre-charged with a medicament or other treatment liquid, this means that the actuating rod has to project from the reservoir from the time of pre-charging until the time of use. If the device is to be carried by the user for example for use only when an emergency arises, the projecting rod generates a potential for damage to the device as a whole. Of course, if the device is for emergency use, then any risk of damage to the device has to be minimised as has any risk of accidental discharge of the treatment liquid

International Patent Application PCT/US02/05114, is directed at an operating mechanism for the plunger in a dosage delivery device of the kind referred to above. The mechanism comprises an actuator slideably mounted with respect to a drive member for driving a plunger within a reservoir. When the device is to be used a dose is set by pulling the actuator along the drive member away from the plunger so as to enable operation of the plunger. The actuator is then pushed and engages the drive member so as to drive the plunger into the reservoir so as to dispense a medicament held therein.

An emergency dosage delivery device of the type described above is generally distributed in a pre-packaged form which is carried around in case of an emergency. Accordingly, it is important that the device in its packaging is robust, compact and easy to remove from its packaging to a configuration in which a medicament it contains can be dispensed. Often, when a device of the invention has to be used, a covering or closure member has first to be removed to expose the discharge opening through which the medicament is to be dispensed. This can be difficult in an emergency situation.

According to the present invention there is provided a dosage delivery device having a housing defining a reservoir; a plunger within the reservoir and movable to decrease the volume of the reservoir so as to discharge material in the reservoir though a discharge opening; and an operating mechanism for the plunger, wherein a closure member seals the discharge opening, and is coupled to the plunger operating mechanism such that it exposes the opening when the mechanism is engaged. In this way, a single movement to prime the mechanism also removes the closure.

The closure member is normally totally removed from the reservoir discharge opening when the device is used. However, it can remain attached, for example pivotally attached, either to the reservoir housing, or to the coupling with the operating mechanism.

The device according to the present invention can also be adapted such that the extension or priming of the operating mechanism for the plunger is effected by a resilient force released by a catch when the device is to be used. This step can, of course, be coupled to the removal of a discharge orifice closure, as discussed above. The resilient force can be provided by a spring compressed between the elements of the operating mechanism.

With a mechanism according to the invention attached to the reservoir of a dosage delivery device, the length of the coupled elements extending from the reservoir is reduced relative to that of a single operating rod by a factor at least equal to the number of elements in the mechanism. Typically there are two elements in a mechanism according to the invention, but more could be used. The elements will normally be telescopically engaged, to provide good stability when extended.

In order to operate the plunger in a dosage delivery device, the extended elements in a mechanism according to the invention must be held in their extended state. This is conveniently accomplished by a simple latch mechanism. As the mechanism is extended, for example by withdrawal of the distal element, a latch on one element engages a recess on the other, enabling the distal element to lock with the proximal element for the return movement forcing the plunger towards the delivery device discharge opening. In an alternative arrangement, the distal element can be formed with a key that is turned into a recess in the proximal element when the elements are in their extended states. This arrangement has the advantage that it can be easier to re-use the unit, if re-use is desired.

The wastage of medicaments in delivery devices of the type referred to above where a plunger is used to discharge liquid from a reservoir through a discharge opening can be a problem. In the region of the discharge opening there is often a chamber just upstream of the opening from which liquid is not discharged with the consequence that this amount of liquid has to be disposed of elsewhere, without being used. It is proposed to design the plunger in a liquid dosage delivery device in such a manner that its shape on the side facing the opening is matched to the internal profile of the discharge opening. Thus, where the reservoir merges with a frustro-conical section at the discharge opening the side of the plunger facing the discharge opening is also made frustro-conical. In another arrangement, a spigot is formed on the side of the plunger facing the discharge opening to match a cylindrical passageway extending from the reservoir to the discharge opening itself.

If the operating mechanism is inadvertently retracted during or after the medicament is dispensed from the reservoir, the medicament can be sucked back into the reservoir meaning that an incorrect dose is dispensed to the patient. To avoid this in a preferred embodiment of the present invention the operating mechanism is configured so that when the operating mechanism is depressed in the locked extended state the plunger is moved so as to decrease the volume of the reservoir whereas when the operating mechanism is retracted the plunger is unaffected. This can be achieved by arranging the coupling elements to engage when the operating mechanism is depressed and to disengage when the operating mechanism is retracted.

The invention will now be described by way of example and with reference to the accompanying schematic drawings wherein:

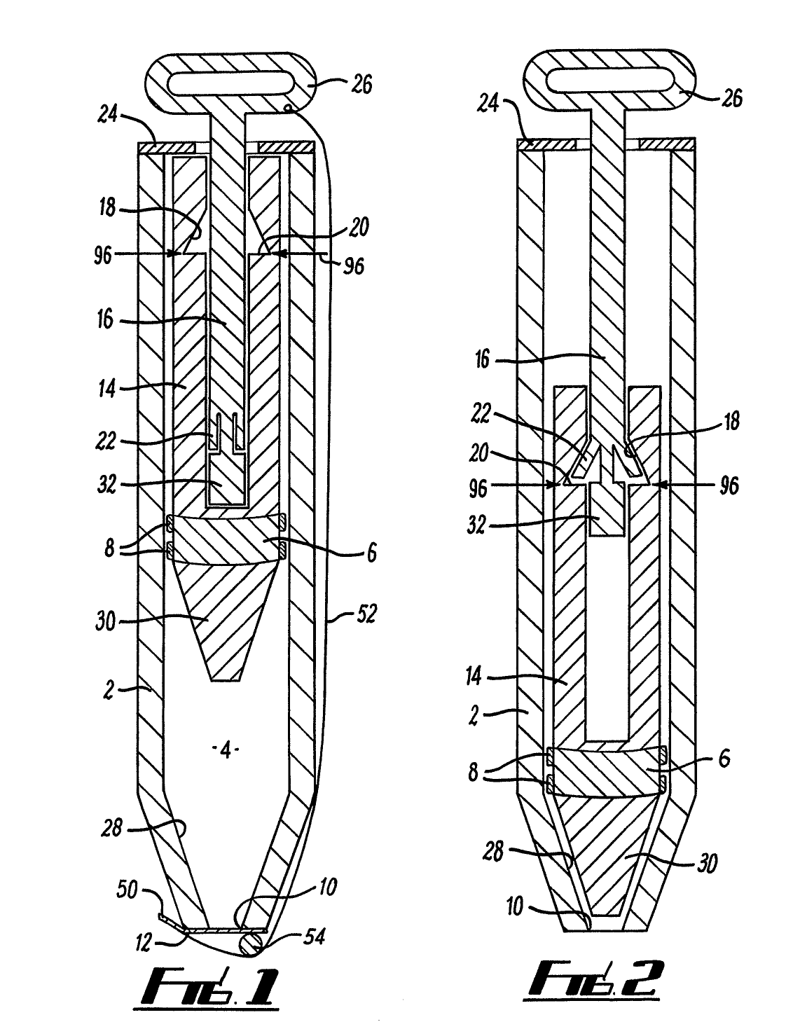

FIG. 1 is a longitudinal sectional view of a dosage delivery device prior to use according to the present invention;

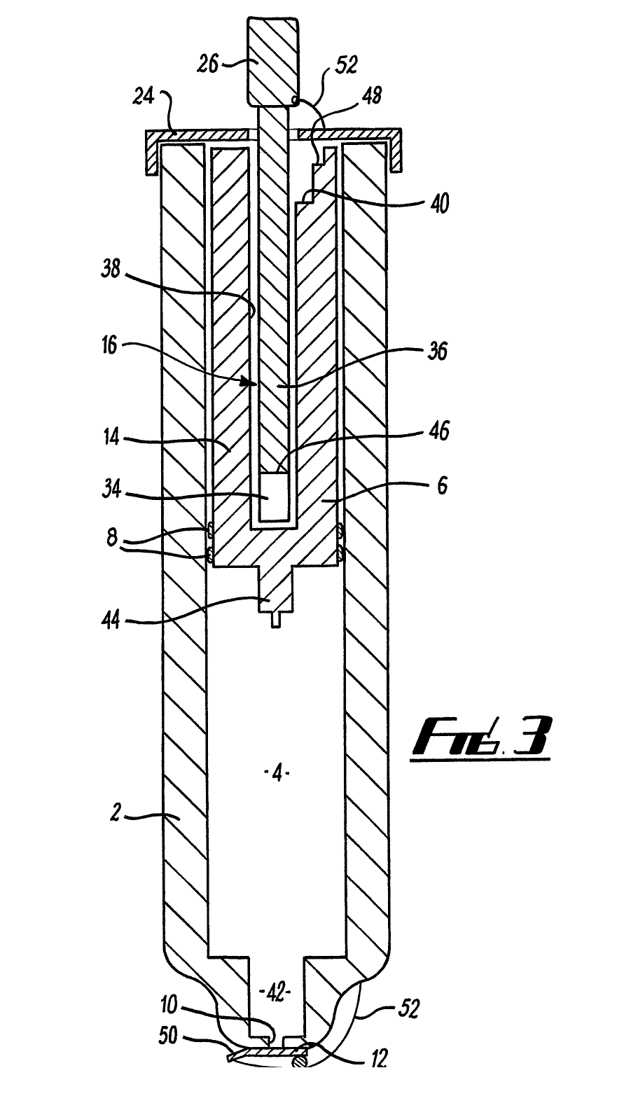

FIG. 2 is a view similar to that of FIG. 1 showing the device after use;

FIG. 3 is a view similar to that of FIG. 1 of an embodiment of the present invention;

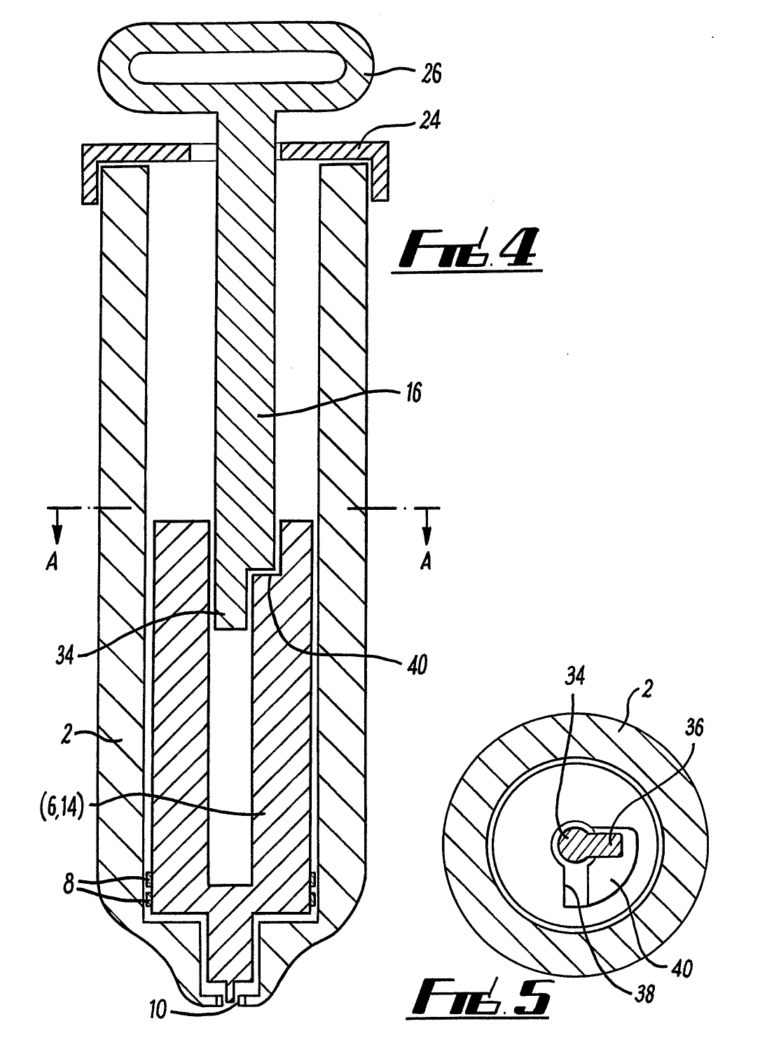

FIG. 4 is a view similar to that of FIG. 3, but showing the device after use;

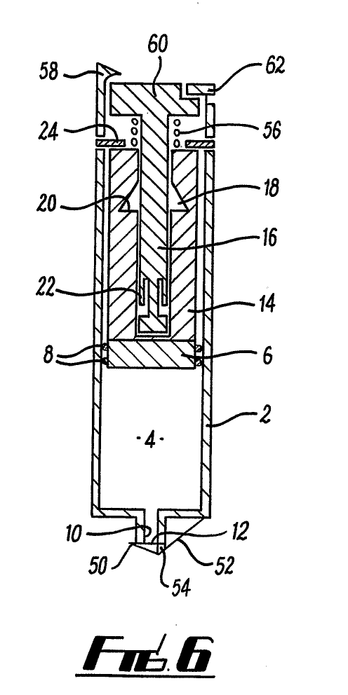

FIG. 5 is a cross-sectional view taken along the line of FIG. 4;

FIG. 6 shows a further embodiment of the present invention utilising a delivery device of the type shown in FIGS. 1 and 2;

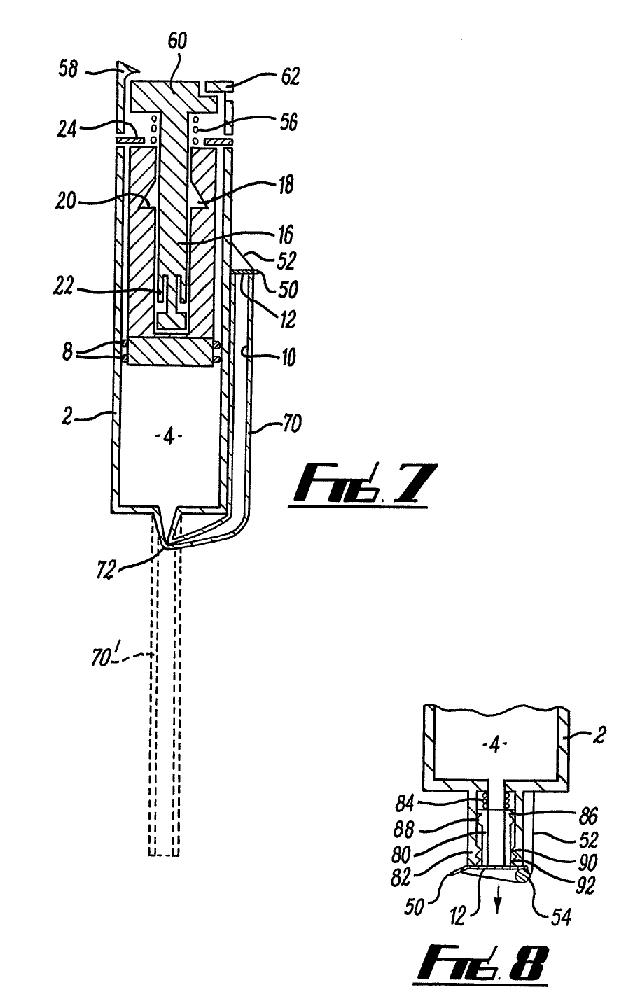

FIG. 7 shows a further embodiment of the present invention for administering a medicament anally; and

FIG. 8 shows an alternative embodiment of the present invention for administering a medicament anally.

The device shown in FIG. 1 comprises a generally cylindrical housing 2 defining a reservoir 4 for medicament. A plunger 6 is fitted within the reservoir with seals 8 closing the upper boundary of the reservoir as shown. At the lower end is formed a discharge opening 10 sealed by an adhesive strip 12.

At the upper end of the housing as shown, is installed an operating mechanism for the plunger 6. The mechanism comprises two elements 14 and 16, with the element 16 telescopically engaged in the element 14. The element 14 has a generally cylindrical cross-section, but is formed with diametrically opposed recesses 18 in its inner wall which define shoulders 20. At the proximal end of the element 16 relative to the plunger 6, are formed a pair of arms 22 which are normally biased resiliently outwardly relative to the axis of the element 16, but constrained from so doing by the inner wall of the element 14.

At the end of the housing distal from the discharge opening 10, a plate 24 holds the element 14 between the plunger 6 and the open end of the housing. When the medicament in the reservoir 4 is required, the element 16 is withdrawn from the element 14 by pulling on the ring 26, until the arms 22 reach the recesses 18. The user can of course recognise when this takes place, although a further feature can be installed to limit the withdrawal of the element 16 from the element 14.

When a device of the invention has to be used, the strip 12 has first to be removed to expose the discharge opening 10. This can be difficult in an emergency situation. To facilitate its removal, a tab 50 of the strip may be attached to a tie 52 which extends around the opening 10 to the ring 26. In this way withdrawal of the element 16 by pulling on the ring 26 will simultaneously remove the strip 12 to expose the opening 10. The tie may be designed to pull on the tab 50 at an appropriate angle, by a projection 54 (FIG. 1), attached to either the tab 12 or the tie 52. By this means, once the strip 12 is removed, so is the projection 54, and the discharge end of the device is smooth for use.

The element 16 can then be depressed as if it were part of a conventional syringe, and by virtue of the arms 22 engaging the shoulders 20 in the recesses 18, depression of the element 16 will move the plunger 6 towards the opening 10, and discharge liquid from the reservoir, after of course, the strip 12 has been removed. The position of the components after discharge of the medicament from reservoir 4, is shown in FIG. 2.

The housing 2 in the device of FIGS. 1 and 2 defines a generally cylindrical reservoir 4 which converges in a frustro-conical end section 28 towards the discharge opening 10. The plunger 6 on its side facing the discharge opening, is also formed with a frustro-conical end section 30, which matches the profile of the reservoir end section 28. With these matching profiles, when the plunger reaches its lower most position as shown in FIG. 2, the end sections 28 and 30 are complementarily engaged, with only a minimal volume (of medicament) remaining in the reservoir upstream of the opening 10.

The element 16 in the operating mechanism described above for the delivery device consists essentially of a solid rod formed with the expanding arms 22. When the mechanism is assembled, the element 16 is angularly oriented such that the arms 22 are out of alignment with the recesses 20. This enables the elements to be installed in the element 14 with the end 32 assisting in alignment. The end 32 also stabilises the alignment of the element 16 when the elements 14 and 16 are in their extended state as shown in FIG. 2.

In FIGS. 3 to 5 a generally similar device is illustrated, but a different locking mechanism is provided for the elements in their extended state. One of the elements 14 is combined with the plunger 6 to form an integral unit and there is a different profile of the reservoir 4 adjacent discharge opening 10.

In FIGS. 3 to 5 the plunger 6 and element 14 are combined to form a piston movable vertically as shown in the reservoir 4 to discharge medicament therefrom through the opening 10. The element 16 can be described as being in the form of a rod 34 with a lateral flange 36. The flange 36 does not extend the full length of the element 16. At its proximal end relative to the discharge opening, its cross-section is reduced to circular. The element 16 is received in a complimentary recess 38 in the piston (6, 14), as shown in FIG. 3. When needed for use, the element 16 is withdrawn to the point of which it can be rotated to locate the end of the flange 36 on a shoulder 40 formed at the distal end of the piston (6, 14).

As with the embodiment of FIGS. 1 and 2, the tab 50 of the strip 12 may be attached to a tie 52 which extends around the opening 10 to the ring 26. In this way withdrawal of the element 16 by pulling on the ring 26 will simultaneously remove the strip 12 to expose the opening 10.

The element 16 can then be depressed as described above with references to FIGS. 1 and 2, to discharge medicament from the reservoir 4 through the opening 10, after removal of the adhesive strip 12. Once again, an additional mechanism may be included to prevent the total withdrawal of the element 16 from the device. For example, the proximal end of the element 16 may have a lateral extension 46 from the flange 36 which abuts against the plate 24 to prevent the element 16 from being withdrawn from the housing 2, and indicate when it can be rotated to engage the shoulder 40. The extension is received in space 48 when the element 16 is rotated.

The embodiment of FIGS. 3 to 5 has the advantage that the elements of the operating mechanism are of simpler shape and are therefore readily susceptible to plastics moulding techniques. This reduces manufacturing costs, and facilitates manufacture of the devices as disposable items.

Also in the embodiment of FIGS. 3 and 4, the end of the reservoir approximate the discharge opening 10 has a volume 42 of reduced cylindrical cross-section. A spigot 44 at the proximal end of the piston (6, 14) has matching dimensions such that when the piston (6, 14) has moved its full stoke towards the opening 10, the spigot 44 substantially fills the volume 44 of reduced cross-section. Once again then, wastage of medicament is minimised. Additionally, by virtue of the geometry of the matching profiles in this area, slightly greater pressure will be required to complete the discharge of medicament. This provides the user with an indication of when the medicament in the reservoir 4 is close to having been fully discharged.

The embodiment described with reference to FIGS. 3 to 5 is a complete dosage delivery device which can be used for oral or rectal delivery, or coupled to a needle for subcutaneous injections and intravenous delivery systems. For rectal use, the discharge end of the device will be extended to ensure sufficient penetration. The device shown in FIGS. 1 and 2 has the same range of applications, but it will be appreciated that the operating mechanism can be entirely removed, and used separately. Thus, the same basic reservoir structure can be for both hospital and personal emergency use, and for use in a conventional hospital or surgery environment.

It will be recognised that as a pre-charged delivery unit, the device of the present invention can be activated and used extremely swiftly. Indeed, using the tie 52, the entire delivery operation can be effected by a single reciprocal movement of the element 16. This is of self-evident benefit when the device has to be used in emergency situations. It could also have benefit in vaccination programmes, where a single injection can be completed within a very few minutes if not seconds. This also means that an individual being vaccinated can have minimal advance sight of the device, or more particularly a vaccination needle.

The device shown in FIG. 6 comprises a generally cylindrical housing 2 defining a reservoir 4 for medicament. A plunger 6 is fitted within the reservoir with seals 8 closing the upper boundary of the reservoir as shown. At the lower end is formed a discharged opening 10 sealed by an adhesive strip 12.

At the upper end of the housing as shown, is installed an operating mechanism for the plunger 6. The mechanism comprises two elements 14 and 16, with the element 16 telescopically engaged in the element 14. The element 14 has a generally cylindrical cross-section, but is formed with diametrically opposed recesses 18 in its inner wall which define shoulders 20. At the proximal end of the element 16 relative to the plunger 6, are formed a pair of arms 22 which are normally biased resiliently outwardly relative to the axis of the element 16, but constrained from so doing by the inner wall of the element 14.

At the end of the housing distal from the discharge opening 10, a plate 24 holds the element 14 between the plunger 6 and the open end of the housing. The element 16 is held in the element 14 against the force of a spring 56 by a catch 58 that lips over the head 60 of the element 16. The catch 58 is a resiliently flexible component of the housing 2, and when the device is to be used it is pivoted away from the housing, typically by the user's thumb, to release the head 60. The spring 56 then urges the head 60 away from the element 14 to move the elements into their extended state with the arms 22 engaging the recesses 18. The user can then depress the head 60 to move the plunger towards the opening 10, and discharge liquid from the reservoir.

As noted above, the discharge opening is sealed by the strip 12, and this has of course to be removed before the medicament can be discharged. This is accomplished by a tie 52 which couples a tab 50 on the closure member 12 to the initial release of the head 60 of the element 16. The tie 52 extends in a channel (not shown) in the housing 2, to a block 62 attached to the head 60 of the element 16. When the head 60 is released by the catch 58, the block 62 pulls the tie 52 to remove the closure member 12, before detaching itself from the head 60 to enable the element 16 to fully extend relative to the element 14. The tie 52 can of course be otherwise attached to the head 60, but it can be useful to have the block 62 available if for some reason it detached itself before the discharge opening is fully exposed.

In an alternative arrangement (not shown) the closure member is drawn into the channel when the plunger operating mechanism is engaged. This prevents the closure member 12 from hanging loose near the discharge opening 10 at risk of freeing itself altogether (and possibly being swallowed), and also enables the tie to be permanently linked to the head 60, ensuring that the closure member is drawn fully into the channel. The channel can of course be adapted (enlarged) to receive the closure member 12, and to retain it in the event that for some reason the coupling breaks.

The tie 52 extends around a projection 54 at the discharge opening such that it pulls on the tab 50 at an appropriate angle to readily remove the closure member 12. The projection 54 can be an element of the housing 2, but is preferably attached to the closure 12 so that it and the closure member are both removed completely from the opening when the tie 52 is pulled.

The dosage delivery device shown in FIG. 7 is for anal administration of a medicament. The device is similar in structure to that described above with reference to FIG. 6, with like parts identified with like numerals. The difference between the two devices is that the discharge opening 10 is located at the end of an extended nozzle 70, which nozzle extends from the lower end of the reservoir 4. The nozzle 70 is made from a length of resilient tubing material which can be folded over into the position shown in FIG. 7, but which on release of the nozzle, enables the tubing material to resume an undeformed shape as a nozzle which extends directly downwardly from the lower end of the reservoir, as shown by the dotted lines in FIG. 7 and indicated by the reference numeral 70′. In its extended position the nozzle 70′ is sufficiently rigid in an axial direction to enable the nozzle to be inserted into the anus of a patient for the anal delivery of the medicament stored in the reservoir 4.

In its folded position, as shown in FIG. 7, the nozzle 70 is pinched closed at a fold 72 in the resilient tubing forming the nozzle. The fold 72 contains the medicament in the reservoir 4 and prevents it from leaking into the length of the nozzle 70 beyond the fold 72. It is not essential to have a fold 72 that blocks the nozzle 70 and in an alternative embodiment, the medicament can be contained in the reservoir 4 and in the nozzle 70 prior to use of the device. The nozzle 70 is bent around the lower end of the reservoir 4 so that it lies alongside the housing 2 in a folded position, as shown in FIG. 7. The nozzle is held in the folded position by the closure member 12 and the tie 52, which tie is connected to the block 62 or directly to the head 60 in a similar manner as is described above in relation to FIG. 6. An additional form of breakable attachment, for example a strip of adhesive paper can be used to secure the nozzle to the housing in its folded position. The breakable attachment is broken when the dosage device is to be used.

As described above, the discharge opening 10 is sealed by the strip 12, and this is removed by a tie 52 which couples a tab 50 on the closure member 12 to the head 60 of the element 16. The tie 52 extends in a channel (not shown) in the housing 2, to a block 62 attached to the head 60 of the element 16. When the head 60 is released by the catch 58, the block 62 pulls the tie 52 to remove the closure member 12, before detaching itself from the head 60 to enable the element 16 to fully extend relative to the element 14. As the closure member 12 is removed from the discharge opening 10, the nozzle 70, because it is made up of a piece of resilient tubing takes up its unfolded shape 70′ shown by dotted lines in FIG. 7. This enables the medicament from the reservoir 4 to be dispensed anally.

By having the nozzle 70 in a folded position prior to use, the space required to store the dosage delivery device of FIG. 7 is reduced by the length of the nozzle 70. This means that the packaging for the device can be made smaller and so is more convenient to store and to carry around in case of an emergency.

In the arrangement in FIG. 8, a nozzle extending from the lower end of the reservoir 4 is formed in two parts 80, 82 which are stored in a contracted position with the part 80 telescoped within the part 82. The distal end of the outer and inner parts 80, 82 are covered by a closure member 12, of the type described above which is attached to a tie 52. The closure member 12 holds the inner part 80 telescoped within the outer part 82 against the force of a biasing spring element 84. When the operating mechanism for a plunger of the device is actuated, the closure member 12 is removed from the end of the part 82 and the spring element 84, shown in FIG. 8 as a helical spring, pushes the inner part 80 of the nozzle to the distal end of the outer part 82 of the nozzle. The inner part 80 of the nozzle is formed on its outer surface with an annular stop 86 and an annular latch member 88. The outer part 82 of the nozzle is formed with an adjacent pair of annular latch members 90, 92 for catching the latch element 88 formed on the inner part of the nozzle 80. Thus, when the inner part 80 of the nozzle is moved to the distal end of the outer part 82 of the nozzle under the biasing force of the spring element 84, the latch element 88 on the inner part 80 is caught between the pair of latch elements 90, 92 on the outer part 82 so as to secure the inner part in an extended position in which all but the proximal end of the inner part 80 extends beyond the distal end of the outer part 82 so as to form a nozzle which is long enough to administer the medicament in the reservoir 4 anally. The stop 86 formed on the inner part 80 of the nozzle abuts the latch element 90 to maintain the inner part 80 of the nozzle within the outer part 82 of the nozzle.

Again the device of the embodiment of FIG. 8 has the advantage of reducing the size of the packaging required for the device, making it easier to store and more convenient to carry around.

With reference to FIGS. 1 and 2 the two elements 14, 16 which make up the operating mechanism for the plunger 6 can be formed so that after the operating mechanism is engaged, withdrawal of the element 16 does not cause withdrawal of the plunger. This can be important to ensure that inadvertent withdrawal of the element 16 does not cause medicament already dispensed to be sucked back into the reservoir 4, for example on withdrawal of the dosage delivery device from the patient. To this end the element 14 may be cut off at the level indicated by the arrows 96, so that the element 14 extends from the plunger 6 only as far as the shoulders 20 with the material beyond the shoulders being removed.

Once the element 16 has been withdrawn to the position with respect to the element 14 in which the pair of arms 22 move outwardly, then depression of the element 16 will cause the arms 22 to engage the shoulders 20 of the element 14 causing the element 14 to depress the plunger 6. However, with the material above the level 96 removed, if at any point after the depression of the element 16 has begun in this way, if the element 16 is inadvertently withdrawn the arms of the element 16 will disengage the shoulders 20 of the element 14 and the element 14 will remain in place. Therefore, the plunger 6 is not withdrawn by such a withdrawal of the element 16.

This feature is also present in the embodiments of FIGS. 3 to 5. When the element 16 is rotated into the locked position, depression of the element 16 causes depression of the element 14 by engagement of the lateral extension 46 with the shoulder 40 so as to thereby depress the plunger 6 so as to decrease the volume of the reservoir 4. However, when in the locked position, if the element 16 is retracted from the housing 2, the element 16 disengages the element 14 and so the element 14 and thus the plunger are unaffected by the retraction of the element 16.

1-17. (canceled)

18. A dosage delivery device comprising:
a housing defining a reservoir with a discharge opening;
a plunger within the reservoir and movable to decrease the volume of the reservoir so as to discharge material in the reservoir via the discharge opening; and
an operating mechanism for the plunger comprising coupled elements movable relative to each other between contracted and extended states and a locking arrangement for locking them in the extended state;
wherein the operating mechanism is attached to the reservoir in the contracted state, and is movable to the extended state to enable operation of the plunger and wherein a closure member seals the discharge opening, and a coupling is provided for coupling the closure member to the plunger operating mechanism such that it exposes the discharge opening when the mechanism moves to the extended state.

19. The dosage delivery device according to claim 18 further comprising a catch for holding the elements of the operating mechanism in their contracted state against a resilient force wherein operation of the catch releases the elements to move to their extended state.

20. The dosage delivery device according to claim 18 further comprising a catch for holding the elements of the operating mechanism in their contracted state against a resilient force, wherein operation of the catch releases the elements to move to their extended state and a spring is compressed between the elements of the operating mechanism for providing the resilient force.

21. The dosage delivery device according to claim 18, wherein the elements of the operating mechanism are telescopically engaged and the device additionally comprises a catch for holding the elements of the operating mechanism in their contracted state against a resilient force, wherein operation of the catch releases the elements to move to their extended state and a coil spring surrounds an inner element of the operating mechanism and engages an end of the outer element of the operating mechanism, which spring is compressed between the elements of the operating mechanism for providing the resilient force.

22. The dosage delivery device according to claim 18, wherein the closure member remains attached to the reservoir after exposure of the opening.

23. The dosage delivery device according to claim 18, wherein the closure member remains pivotally attached to the reservoir after exposure of the opening.

24. The dosage delivery device according to claim 18, wherein the coupling between the closure member and the plunger operating mechanism totally removes the closure member from the opening when the mechanism elements move to their extended state.

25. The dosage delivery device according to claim 18, wherein the coupling mechanism comprises a tie extending round a projection to an edge of the closure member to pull the closure member away from the opening when the operating mechanism elements move to their extended state.

26. The dosage delivery device according to claim 18, wherein the coupling mechanism comprises a tie extending round a projection to an edge of the closure member to pull the closure member away from the opening when the operating mechanism elements move to their extended state and wherein the projection is attached to the closure member.

27. The dosage delivery device according to claim 18, wherein the coupling with the closure member detaches from the plunger operating mechanism when the reservoir opening is exposed.

28. The dosage delivery device according to claim 18, wherein a channel is formed in the reservoir housing and the coupling between the closure member and the plunger operating mechanism runs along the channel.

29. The dosage delivery device according to claim 18, wherein a channel is formed in the reservoir housing and the coupling between the closure member and the plunger operating mechanism runs along the channel and wherein the coupling with the closure member draws the closure member into the channel when the plunger operating mechanism is engaged.

30. The dosage delivery device according to claim 18 further comprising a resilient nozzle extending from a lower end of the reservoir with the discharge opening located at a distal end of a resilient nozzle, wherein the closure member holds the nozzle in a folded position such that when the closure member is detached from the discharge opening of the nozzle, the nozzle adopts an unfolded position suitable for anal administration of a medicament from the device.

31. The dosage delivery device according to claim 18 further comprising a spring biased telescoping nozzle arrangement extending from a lower end of the reservoir with the discharge opening located at the distal end of the nozzle, wherein the closure member covers and holds the discharge opening in a contracted position such that when the closure member is detached, the telescoping nozzle extends into a position suitable for anal administration of a medicament from the device.

32. The dosage delivery device according to claim 18, wherein when the operating mechanism is depressed in the locked extended state the plunger is moved so as to decrease the volume of the reservoir whereas when the operating mechanism is retracted the plunger is unaffected.

33. The dosage delivery device according to claim 18, wherein when the operating mechanism is depressed in the locked extended state the coupled elements engage and the plunger is moved so as to decrease the volume of the reservoir whereas when the operating mechanism is retracted the coupled elements disengage and the plunger is unaffected.
